(19) United States
(12) Patent Application Publication
(10) Pub. No.: US 2024/0032823 A1
(43) Pub. Date: Feb. 1, 2024

(54) TEXTILE COMPUTING SYSTEM WITH DISTRIBUTED ARCHITECTURE

(71) Applicant: MYANT INC., Toronto (CA)

(72) Inventors: Tony CHAHINE, Toronto (CA); Godfried Gysbrecht EDELMAN, Toronto (CA); Amin MAHNAM, Toronto (CA)

(21) Appl. No.: 18/039,903

(22) PCT Filed: Dec. 6, 2021
(86) PCT No.: PCT/CA2021/051744

§ 371 Date: Jun. 1, 2023

Related U.S. Application Data
(60) Provisional application No. 63/121,698, filed on Dec. 4, 2020.

(51) Int. Cl. A61B 5/11; A61B 5/00

(52) U.S. Cl. CPC A61B 5/112; A61B 5/0024; A61B 5/6828; A61B 5/6829; A61B 5/7405

(57) ABSTRACT

A textile computing platform and method of operating the same. The platform includes at least one textile and at least one computing node including a sensory device positioned at the at least one textile. The platform includes a gateway device coupled to the computing node. The gateway device may be configured to: transmit, via a textile communication network, a discovery signal and a network time protocol signal; receive node discovery data from computing nodes to enumerate a subset of computing nodes for a current textile system state; generate physiological analytics based on a series of sensory data records received from the enumerated subset of computing nodes, the respective sensory data records associated with a time-stamp based on the network time protocol signal; and transmit actuating signals to the one or more enumerated subset of computing nodes for generating feedback to a user on a substantially real time basis.

### CROSS REFERENCE TO RELATED APPLICATIONS

This application claims priority from U.S. provisional patent application No. 63/121,698, entitled “TEXTILE COMPUTING SYSTEM WITH DISTRIBUTED ARCHITECTURE”, filed on Dec. 4, 2020, the entire contents of which are hereby incorporated by reference herein.

### FIELD

Embodiments of the present disclosure generally relate to textile computing systems, and in particular to textile computing systems with distributed architecture.

### BACKGROUND

A textile computing platform may include a collection of devices, such as sensors, actuators, control systems, among other examples, for monitoring users, conducting analytics, or for providing notification output to users. The respective devices of the textile computing platform may be integrated devices or discrete devices.

### SUMMARY

Embodiments of textile computing systems with distributed architecture are described in the present disclosure.

Textile computing systems may include one or more textile garments configured to be worn by a user. In some embodiments, textile garments may include one or more computing nodes for interfacing with anatomical features of a user. Computing nodes may include a processor and one or more sensor or actuator devices coupled to the processor. Embodiments of computing nodes may be configured to generate data sets based on the one or more sensors and may be configured to generate output via actuator devices. In some scenarios, the computing nodes may operate as discrete computing units. In some other scenarios, the computing nodes may operate in concert or in combination with a plurality of other computing nodes for generating data sets representing physiological data associated with a user or for providing feedback to a user (e.g., thermal feedback, haptic feedback, etc.). In some embodiments, a plurality of computing nodes may be interconnected based on electrically conductive textile fibers (e.g., wired network) or wirelessly (e.g., wireless communication network, such as Bluetooth Low Energy (BLE)).

When textile garments are worn by a user, the computing nodes may be positioned proximal to an anatomical feature of the user. For example, a long-sleeve shirt may include one or more computing nodes positioned along a sleeve to detect skin temperature or a user's pulse, when the long-sleeve shirt is worn by a user. In another example, a sock may include one or more computing nodes for interfacing with toes or with an arch of a user's foot, when the sock is worn by a user.

In some embodiments, the textile computing systems may be configured to generate physiological insights associated with the user based on the one or more sensor devices positioned by the textile garments proximal to anatomical features of the user. In some embodiments, the textile computing systems may provide physiological insights as feedback to a user on a substantially real-time basis or on a summary basis on an on-demand basis. As an example, actuator devices may be positioned by textile garments proximal to anatomical features of a user and may be configured to provide at least one of haptic feedback, thermal feedback, or acoustic feedback, among other examples. Such feedback may be provided to assist the user during physical activity to guide the user with movement form, to alert the user of physiological status (e.g., temperature status, hydration status, etc.).

In some embodiments, the textile computing systems may be configured to generate physiological insights based on detection of the computing node type, quantity, or positioning proximal to a user's anatomical features. For example, to generate gait analysis, it may be a prerequisite for the textile computing system to include one or more sensors proximal to a user's foot, lower leg, knees, or upper leg. In another example, to generate thermal regulation analysis, it may be a prerequisite for the textile computing system to include one or more sensors proximal to a user's upper body region. In scenarios, example textile computing systems may generate physiological insights when an identified minimum number of computing nodes may be detected or enumerated.

In some scenarios, the quantity or type of textile garment worn by a user may be based on environmental conditions (e.g., temperature, humidity, etc.) or based on an anticipated activity (e.g., running versus swimming, etc.). Some embodiments of textile computing systems may be configured to conduct discovery operations for enumerating available textile garments, thereby determining the quantity, type, or positioning, among other system configurations, of computing nodes. By conducting discovery operations, embodiments of textile computing systems may be configured to provide physiological insights based on dynamically enumerated computing nodes.

In scenarios where a limited number of computing nodes may be positioned about the user's physiological features, the textile computing system may be configured to learn about what computing nodes are available, learn the computing node capabilities, or learn positioning of the computing nodes about the user's anatomical features (e.g., whether computing nodes are positioned at user's foot, user's knees, user's arm, among other examples). By learning computing node capabilities, example textile computing devices may be configured to adaptively generate physiological insights based on available user physiological data.

In some embodiments, textile garments may include a plurality of nodal regions and a plurality of computing nodes respectively coupled to a nodal region of the textile. The respective computing nodes associated with a nodal region of the textile may be in communication with at least one other computing node via an interconnection bus.

The respective computing nodes may be configured for distributed sensory data acquisition by generating sensor data substantially independently of other computing nodes. Based on sensory data acquisition by the distributed plurality of computing nodes, one or more of the computing nodes may determine a modality or change in modality associated with the textile worn by a user. Based on the modality or modality change, the respective distributed computing nodes may provide actuating feedback output via actuating structures in concert with other distributing computing nodes.

In some embodiments, data transmission among computing nodes in a plurality may be based at least in part on a wireless interconnection bus (e.g., via a Bluetooth™ mesh-based network) and a wired interconnection bus (e.g., conductive fiber network integral to the textile). In scenarios where computing nodes may be physically distant or may be separated by numerous intermediary computing nodes, the plurality of computing nodes may conduct operations to determine a most efficient path for data transmission among the computing nodes. In some scenarios, wireless mesh-based interconnection buses may provide computing nodes with flexibility to dynamically relay data transmissions to other computing nodes associated with interconnected textiles.

In some embodiments, textile computing systems disclosed herein may be implemented such that two or more textiles (e.g., textile garments) may be interconnected for data or power transmission. Plurality of computing nodes among a plurality of textiles may be interconnected based at least in part on: (i) a wireless interconnection bus (e.g., via a Bluetooth™ mesh-based network, among other mesh-based network examples); or (ii) a wired interconnection bus (e.g., conductive fiber network integrated in respective textiles. Such embodiments of textile computing systems may be beneficial for providing dynamic expansion of a textile computing system based on modular peer-to-peer discovery of textiles having computing nodes thereon.

In an aspect, the present disclosure describes a textile computing platform. The textile computing platform may include at least one textile including a plurality of nodal regions for interfacing with one or more anatomical features of a user, the respective nodal regions configured to be in communication with at least one nearby nodal region; at least one computing node positioned substantially within one of the textile nodal regions, the at least one computing node including at least one sensory device; and a gateway device coupled to the plurality of textile nodal regions. The gateway device may include: a gateway processor; and a memory coupled to the gateway processor and storing processor-executable instructions that, when executed by the gateway processor, configure the gateway processor to: transmit, via a textile communication network, a discovery signal and a network time protocol signal; receive node discovery data from one or more computing nodes to enumerate a subset of computing nodes for a current textile system state; generate physiological analytics based on a series of sensory data records received from the enumerated subset of computing nodes for the current textile system state, the respective sensory data records associated with a time-stamp based on the network time protocol signal; and transmit actuating signals to the one or more enumerated subset of computing nodes based on the physiological analytics for generating feedback to a user on a substantially real time basis.

In some embodiments, the enumerated subset of computing nodes may include one or more actuating devices, and wherein the transmitted actuating signals are configured to generate a combination output at the one or more actuating devices.

In some embodiments, the node discovery data may include data records identifying a sensor device type and a sensor position relative to an anatomical feature of the user.

In some embodiments, the processor-executable instructions, when executed by the gateway processor, configure the gateway processor to: determine a subset of physiological analytics that correspond to at least one of the identified sensor device type or a sensor position, wherein the subset of physiological analytics is based on capabilities of sensory data records associated with the identified sensor device type or sensor position.

In some embodiments, the physiological analytics may include at least one of gait analysis, biofeedback training, auto-coaching operations in substantial real-time, gamification operations, health monitoring, or medical diagnosis operations.

In some embodiments, the series of sensory data records may include at least one of inertial measurement unit data, temperature or heat flux data at a skin surface of the user, or electrical biosignals across a skin surface of the user.

In some embodiments, wherein the plurality of computing nodes may be positioned at positions across the anatomical features of the user for a defined physiological analytics type.

In some embodiments, the plurality of computing nodes may be positioned across at least one of a foot of the user, a position proximal to a knee of the user, or a position proximal upper leg or lower leg muscles of the user for generating gait analysis for the user.

In some embodiments, the at least one computing nodes may respectively include: a node processor; the at least one sensory-actuating device coupled to the node processor; and a node memory coupled to the processor and storing processor-executable instructions that, when executed by the node processor, configure the node processor to: generate sensory data records based on the at least one sensory-actuating device and associating a time-stamp based on a received network time protocol signal; receive one or more actuating signals for generating sensory-actuating output for application across an anatomical feature of the user as a combination output of the enumerated subset of computing nodes.

In some embodiments, the at least one sensory-actuating device may be configured to provide at least one of haptic output, heating or cooling output, electrical stimulation, or acoustic output across the anatomical feature of the user.

In some embodiments, the node memory may include processor-executable instructions that, when executed by the node processor, configure the node processor to: generate physiological analytic portions based on the sensory data records generated at the present computing node, the physiological analytic portions being subset analytics for generating physiological analytics based on a combination of computing nodes of the textile computing system.

In some embodiments, the textile communication network may include a combination of a conductive fibers integral to the at least one textile and wireless communication circuits for wireless communication.

In some embodiments, at least one of the plurality of nodal regions may include a nodal receptacle adapted to removably receive a discrete computing node therein.

In some embodiments, at least one computing node may include a power source coupled to the node processor, and wherein the textile communication network is configured to transmit power to adjacent computing nodes associated with nearby nodal regions.

In another aspect, a method for a textile computing system may include at least one textile for interfacing with one or more anatomical features of a user and at least one computing node including a sensory device positioned on the at least one textile. The method may include: transmitting, via a textile communication network, a discovery signal and a network time protocol signal; receiving node discovery data from one or more computing nodes to enumerate a subset of computing nodes for a current textile system state; generating physiological analytics based on a series of sensory data records received from the enumerated subset of computing nodes for the current textile system state, the respective sensory data records associated with a time-stamp based on the network time protocol signal; and transmitting actuating signals to the one or more enumerated subset of computing nodes based on the physiological analytics for generating feedback to a user on a substantially real time basis.

In some embodiments, the enumerated subset of computing nodes may include one or more actuating devices, and the transmitted actuating signals may be configured to generate a combination output at the one or more actuating devices.

In some embodiments, the node discovery data includes data records identifying a sensor device type and a sensor position relative to an anatomical feature of the user.

In some embodiments, the method may include: determining a subset of physiological analytics that correspond to at least one of the identified sensor device type or a sensor position, where the subset of physiological analytics may be based on capabilities of sensory data records associated with the identified sensor device type or sensor position.

In some embodiments, the plurality of computing nodes may be positioned across at least one of a foot of the user, a position proximal to a knee of the user, or a position proximal upper leg or lower leg muscles of the user for generating gait analysis for the user.

In some embodiments, wherein the series of sensory data records may include at least one of inertial measurement unit data, temperature or heat flux data at a skin surface of the user, or electrical characteristics across a skin surface of the user.

In various aspects, the disclosure provides corresponding systems and devices, and logic structures such as machine-executable coded instruction sets for implementing such systems, devices, and methods.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many features and combinations thereof concerning embodiments described herein will appear to those skilled in the art following a reading of the present disclosure.

### DESCRIPTION OF THE FIGURES

In the figures, embodiments are illustrated by way of example. It is to be expressly understood that the description and figures are only for the purpose of illustration and as an aid to understanding.

Embodiments will now be described, by way of example only, with reference to the attached figures, wherein in the figures:

FIG. 1 illustrates a textile computing system, in accordance with an embodiment of the present disclosure;

FIG. 2 illustrates an enlarged view of a computing node coupled to a textile, in accordance with embodiments of the present disclosure;

FIG. 3 illustrates a textile computing system, in accordance with another embodiment of the present disclosure;

FIG. 4 illustrates a textile computing platform, in accordance with an embodiment of the present disclosure;

FIG. 5 illustrates a block diagram of a computing device, in accordance with an embodiment of the present disclosure;

FIG. 6 illustrates a textile garment system, in accordance with embodiments of the present disclosure; and

FIG. 7 illustrates a method of a textile computing system, in accordance with embodiments of the present disclosure.

### DETAILED DESCRIPTION

Textile computing systems with distributed architecture are described in the present disclosure. Embodiments of textile computing systems described herein may include a plurality of computing nodes respectively coupled to nodal regions of a textile. Textiles may be one or more garments adapted to be worn by a user. Garments may include facial garments (e.g., masks), shirts, pants, undergarments, hats, among other examples.

When the textile is worn by a user, nodal regions of the textile may be sub-portions of the textile and may be associated with an anatomical region of the user. As an example, the textile may be an undergarment. A sub-portion of the textile may be associated with a waist region of the user. Another sub-portion of the textile may be associated with a thigh or leg region of the user. Other anatomical regions of the user may be contemplated.

In some embodiments, a plurality of computing nodes may respectively be coupled to sub-portions (or nodal regions) of the textile. A computing node may be a discrete operable computing unit for acquiring sensor data (e.g., temperature sensing, among other examples) or for providing actuation (e.g., heating coils, among other examples). A computing node may include a combination of a processor and at least one of a sensor or an actuator, and may conduct operations substantially independently of other computing nodes coupled to other regions of the textile.

As respective computing nodes may conduct operations substantially independently of other computing nodes, the respective computing nodes may detect a sensor data set associated with a corresponding nodal region of the textile (e.g., sensor data associated with a waist region of the user), which may be distinct from another sensor data set associated with another nodal region of the textile (e.g., sensor data associated with a user leg region). Accordingly, a combination of computing nodes conducting operations substantially independently may detect sensor data from or may transmit actuator signals to nodal regions of the computing textile in a distributed way.

In some embodiments, the plurality of computing nodes may conduct operations substantially independently of other computing nodes, and may communicatively interact with other computing nodes for providing an interactive textile computing system. For example, the combination of computing nodes may collectively provide actuation to a user of the textile computing system in concert or as a whole.

In an example garment textile computing system, respective computing nodes may conduct operations substantially independently of other computing nodes for determining physiological status of the garment user at a plurality of anatomical locations. The physiological status at the plurality of anatomical locations may be similar or may be substantially different types of sensor data (e.g., temperature, humidity, among other examples).

The respective computing nodes may transmit sensor data to other computing nodes of the plurality and, in response, may respectively determine an operating modality based at least on the sensor data sets and generate actuation signals adapted to collectively provide actuation in concert (e.g., provide vibratory output in concert). Embodiments of textile computing systems are described herein.

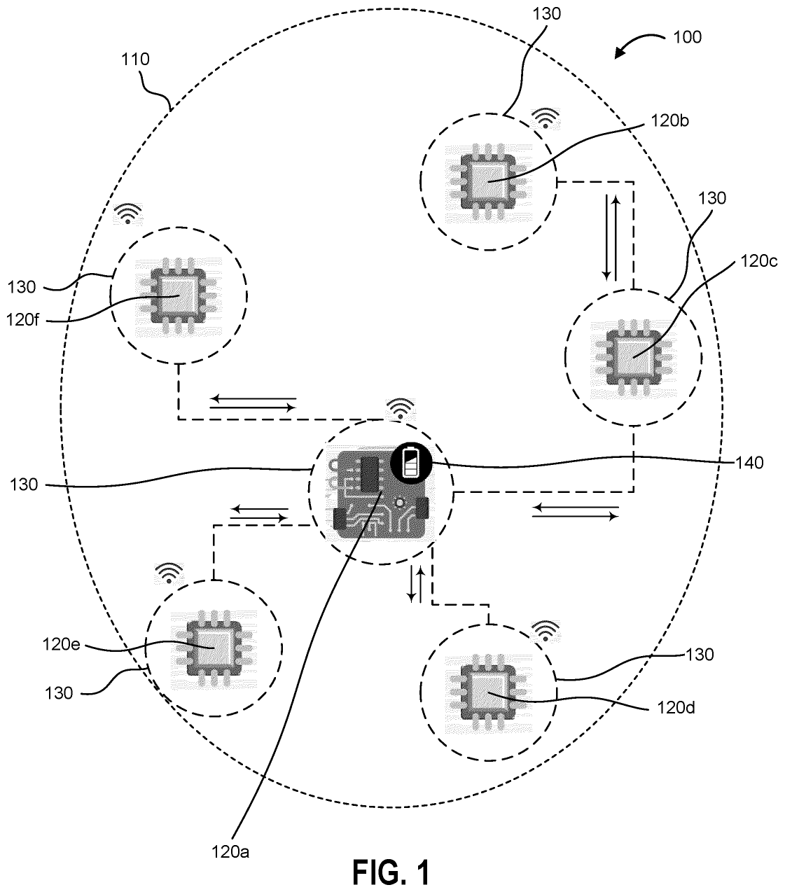

Reference is made to FIG. 1, which illustrates a textile computing system 100, in accordance with embodiments of the present disclosure. The textile computing system 100 may include a textile 110 having a plurality of nodal regions 130. For ease of exposition, the plurality of nodal regions 130 may generally be associated with regions proximal to a computing node. In FIG. 1, the respective nodal regions 130 are illustrated as circular or elliptical shaped regions; however, any other shaped or sized regions may be contemplated.

The textile 110 may be adapted to form a garment. For example, the textile may be an athletic shirt adapted to be worn by a user, and the garment may include a plurality of nodal regions. When the garment is worn by a user, the respective nodal regions may be associated with anatomical features of the user. For instance, a nodal region may correspond to a shirt sleeve, or may correspond to a yoke portion of the shirt for fitting around the neck or shoulders of the user. Other types of garments and nodal regions of the garments may be contemplated.

The textile computing system 100 may include a plurality of computing nodes, identified with reference numerals 120a to 120f. Each of the computing nodes may be respectively coupled to or proximal to a nodal region 130 of the textile 110. Six computing nodes are illustrated in FIG. 1; however, any number of computing nodes respectively coupled to any shapes or positional configuration of nodal regions of the textile 110 may be contemplated.

In some embodiments, the textile 110 may include electrical, mechanical, or electro-mechanical textile structure integrated therein. The textile 110 may include a conductive fiber network coupling two or more nodal regions of the textile 110. In some embodiments, the conductive fiber network may be adapted to provide data or power transmission structures. The conductive fiber network may be configured as a bi-directional bus for transmitting or receiving signals, such as data signals, power signals, or other types of signals that may be carried on the conductive fiber network.

In some embodiments, the textile 110 may be a knitted textile, and may include a plurality of conductive fibers interlaced with a plurality of non-conductive fibers. The conductive fibers may define a plurality of signal paths suitable for delivering data and/or power.

In some embodiments, textile 110 may be formed of other textile forms and/or techniques such as weaving, knitting (warp, weft, etc.) or the like. In some embodiments, textile 110 includes any one of a knitted textile, a woven textile, a cut and sewn textile, a knitted fabric, a non-knitted fabric, in any combination and/or permutation thereof. Example structures and interlacing techniques of textiles formed by knitting and weaving are disclosed in U.S. patent application Ser. No. 15/267,818, entitled “Conductive Knit Patch”, the entire contents of which are herein incorporated by reference.

As used herein “integrated” or “integrally” may include combining, coordinating or otherwise bringing together separate elements so as to provide a harmonious, consistent, interrelated whole. In the context of a textile, a textile can have various sections comprising networks of fibres with different structural properties. For example, a textile can have a section comprising a network of conductive fibres and a section comprising a network of non-conductive fibres. Two or more sections comprising networks of fibres may be said to be “integrated” together into a textile (or “integrally formed”) when at least one fibre of one network is interlaced with at least one fibre of the other network such that the two networks form a layer of the textile. Further, when integrated, two sections of a textile can also be described as being substantially inseparable from the textile. “Substantially inseparable” may refer to the notion that separation of the sections of the textile from each other results in disassembly or destruction of the textile itself.

In some examples, conductive fabric (e.g. group of conductive fibres) can be knit along with (e.g. to be integral with) the base fabric (e.g. surface) in a layer. Such knitting may be performed using a circular knit machine or a flat bed knit machine, warp knit, or the like, from a vendor such as Santoni, Stoll, or Karl Mayer.

The computing nodes (120a to 1200 may respectively include a node processor and at least one of an actuator or a sensor coupled to the node processor. A partial cutaway plan view of a first computing node 120a is illustrated in FIG. 1. The computing node 120a may be based on a printed circuit board.

In some embodiments, the computing nodes (120a to 1200 may include at least one of temperature sensors, humidity sensors, pressure sensors, or chemical sensors (e.g., volatile organic compound sensors, sensors for detecting bio-markers or the like, among other examples). In some embodiments, the respective computing nodes (120a to 1200 may include at least one actuator device or structure configured to alter, in response to sensory data detected at the respective computing node or received from adjacent computing nodes, physical features of the textile. For example, the at least one actuator device or structure may be configured to provide vibratory output, heating or cooling output, acoustic output, among other examples. The actuator device or structure output may be responsive to sensory data detected at one or more computing nodes and may be for altering environmental conditions for the garment user or for alerting the garment user of environment conditions.

In some embodiments, in response to sensory data received from adjacent computing nodes, the respective computing nodes may generate actuator signals for providing actuator output at the respective computing nodes in concert with other computing nodes, such that the combination of actuator output may be provided as a collective output to the garment user.

In some embodiments, the respective computing nodes (120a to 1200 may include a communication circuit for transmitting or receiving signals from one or more other computing nodes of the textile computing platform 100. The communication circuit may be configured for bi-directional signal or power transmission.

For example, the respective computing nodes may be in communication with an adjacent computing node via conductive fibers of the textile 110. The conductive fibers may be configured as an interconnection bus, such that two or more of the computing nodes in the plurality may transmit locally sensed physiological data of a garment user among other computing nodes. Based on dissemination of locally sensed physiological data of the garment users among the plurality of computing nodes, in some embodiments, the respective computing nodes may be configured to generate actuation signals for collectively providing output to the garment user.

As a non-limiting example, one or more computing nodes of a garment textile may include an environmental humidity sensor. One or more other computing nodes of the garment textile may include a temperature sensor. Sensor data sets generated by respective computing nodes may be transmitted among the plurality or adjacent computing nodes, such that the respective computing nodes may be configured to subsequently generate actuation signals to provide, in concert among two or more computing nodes, heating output (e.g., via resistive fibers) to the garment user.

In some embodiments, the communication circuit may be wireless communication circuits, such as Bluetooth™ communication circuits, among other examples. In some embodiments, the communication circuit may be a two-wire (or two-fiber) transmission line for interconnecting computing nodes positioned at respective nodal regions 130 of the textile 110.

In some embodiments, the respective computing nodes may be configured to conduct operations for detecting or discovering adjacent or proximally located computing nodes for sensory data sharing or for actuating signal transmission. In examples where one or more computing nodes may include wireless communication circuits, the respective computing nodes may conduct operations of peer-to-peer discovery to detect proximally located computing nodes for sensory data sharing.

In some embodiments, the respective computing nodes (120a to 1200 may include a power source. In some embodiments, the first computing node 120a may include a battery unit 140, and the battery unit 140 may be configured to provide power to the first computing node 120a. Further, one or more other computing nodes may be configured with a power source, such that the respective computing nodes may have a power source required for conducting operations substantially independently of other computing nodes.

In some embodiments, one or more of the computing nodes may be configured without a power source. For instance, in scenarios where a sixth computing node 120f may not be configured with a power source, the first computing node 120a may be configured to transmit power via conductive textile fibers (e.g., an interconnection bus) to the sixth computing node 120f. In some embodiments, the one or more computing nodes may include power transmission structures that may be inductive charging circuits for wirelessly transmitting power to an adjacent computing node.

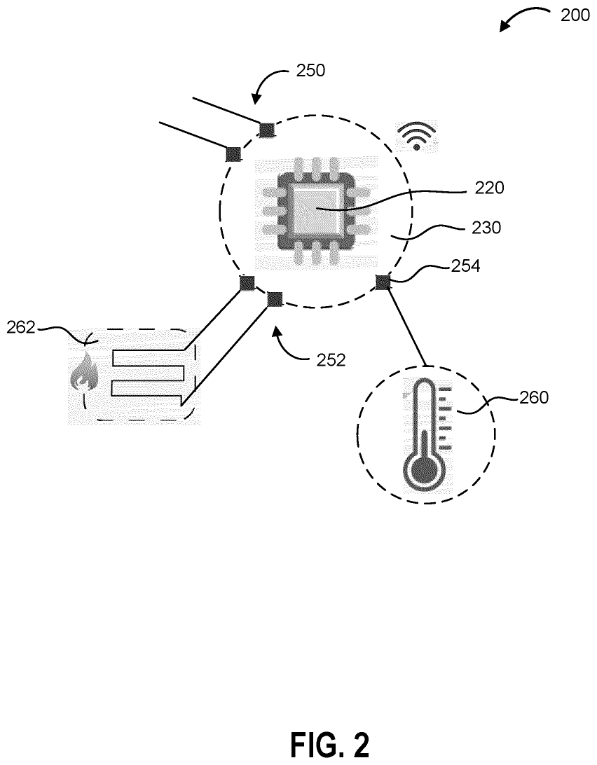

Reference is made to FIG. 2, which illustrates an enlarged view of a computing node 200 coupled to a textile, in accordance with embodiments of the present disclosure. The computing node 200 may be coupled to or positioned at a nodal region 230 of the textile.

The computing node 200 includes a node processor 220. The node processor 220 may be coupled to a communication circuit (not explicitly shown in FIG. 2). In some embodiments, the communication circuit may be configured to transmit or receive data and/or power signals. The communication circuit may be configured to transmit or receive data signals via wireless communication protocols (e.g., Bluetooth™, among other examples).

In some embodiments, the communication circuit may be configured to transmit or receive data and/or power signals via an interconnection bus interface 250 coupled to conductive textile fibers. As a non-limiting example, the interconnection bus interface 250 may be a two-wire interface for coupling the computing node to one or more other computing nodes via a conductive fiber network across the textile. Although a two-wire interface is illustrated in FIG. 2, other implementations of interconnection bus interfaces may be contemplated.

The computing node 200 may include at least one of an actuator structure 262 or a sensor structure 260 coupled to the node processor 220. The sensor structure 260 or the actuator structure 262 may be textile structures integral to the textile therein. For example, conductive paths or structures may be integrated into textiles by one or a combination of methods including inlaying, knitting, weaving, embroidery, adhesive bonding, or mechanical bonding.

In some embodiments, the sensor structure 260 may be a textile sensing structure, such as a pressure sensing structure, a biometric sensing structure, or a physiological sensing structure, among other examples, for receiving touch controls, for detecting motion, for detecting physiological data of a user, or other type of data. In some embodiments, physiological data may include electrophysiological metrics such as heart rate variability, hydration metrics, respiration metrics, or the like for detecting a user physiological state. In some examples, physiological data may include sensed data relating to biomechanics, hemodynamics, biochemical, or electrophysiology.

In some embodiments, the actuator structure 262 may be configured to provide actuating output, such as temperature change (e.g., heating or cooling effect), haptic output, visible output, among other examples.

In the illustrated example of FIG. 2, the sensor structure 260 is enlarged for ease of exposition, and may be coupled to the computing node 200 via a conductive textile input interface 254. The sensor structure 260 may include a combination of textile fibers configured to provide environment temperature sensing capability based on expansion or contraction fiber properties. Other examples of sensing capability may be contemplated. For example, the node processor 220 may be configured to detect, based on the sensor structure 260, sensory data associated with electrocardiogram signals, electroencephalogram signals, photoplethysmogram signals, or other signals. The sensor structure 260 may be configured for targeted sensory data types based at least in part on what anatomical region the sensor structure 260 may be positioned. For example, when detecting ECG signals, the sensor structure 260 may be associated with nodal regions of a textile garment that may correspond to a user's wrist or neck for detecting a user pulse or heart beat.

Further, the actuator structure 262 of FIG. 2 is enlarged for ease of exposition, and may be coupled to the computing node 200 via a conductive textile output interface 252. The actuator structure 262 may be a series of resistive fibers integrated in the textile and, in response to sensory data detected based on the sensor structure 260 or based on sensory data received from other computing nodes, be configured to deliver heating at the nodal region of the textile.

In some embodiments, the computing node 200 may include the sensor structure 260 to the exclusion of the actuator structure 262, and the node processor 220 may transmit detected sensory data to other computing nodes 200 via the interconnection bus interface 250. In some embodiments, the adjacent or other computing nodes 200 may, subsequently, be configured to generate actuator signals for providing actuator output based on the above-described detected sensory data.

Conversely, in some embodiments, the computing node 200 may include the actuator structure 262 to the exclusion of the sensory structure 260, and the node processor 220 may receive sensory data from other computing nodes 200 via the interconnection bus interface 250. In the present example, the node processor 220 may generate actuator signals for activating the actuator structure 262 to provide actuator output to a user of the textile.

In some scenarios, a plurality of computing nodes may conduct operations substantially independently of other computing nodes for detecting sensory data. In response to transmitting detected sensory data to adjacent or other computing nodes via interconnection busses, the plurality of computing nodes may be configured to determine an operating modality based on the collective sensor data sets and generate actuation signals adapted to collectively provide actuation output in concert at the respective textile nodal regions.

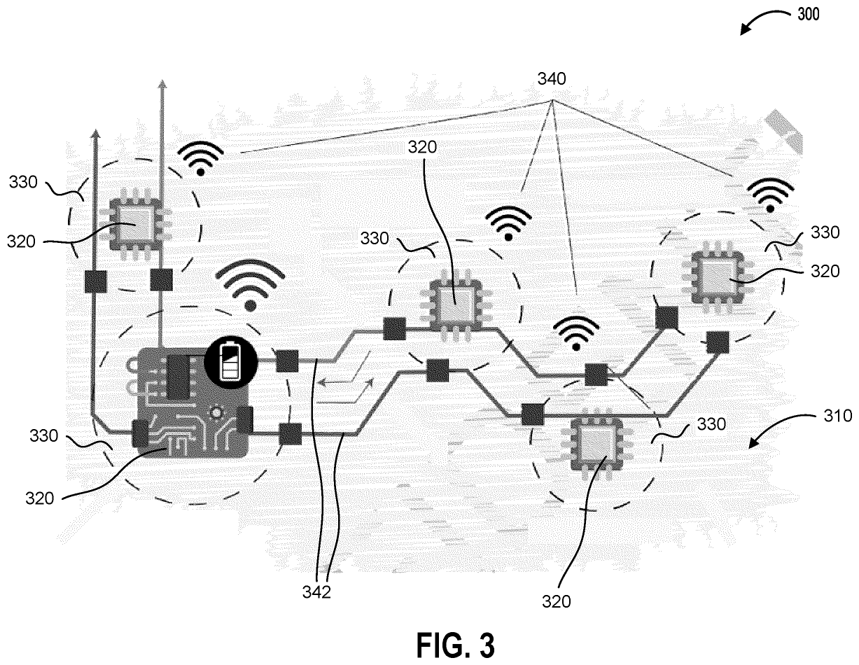

Reference is made to FIG. 3, which illustrates a textile computing system 300, in accordance with another embodiment of the present disclosure. In FIG. 3, the textile computing system 300 may include a textile 310. For ease of exposition to illustrate features of the present disclosure, the textile 310 may be a foot garment, such as a sock.

The textile 310 may include a plurality of computing nodes 320 respectively coupled to a nodal region 330 of the textile 310. The illustrated nodal regions 330 are illustrated as circular regions for convenience; however, the respective nodal regions 330 may be associated with other shapes or configurations and may be without precisely delineated or specific boundaries.

The respective computing nodes 320 may include a node processor and at least one of a sensor structure or an actuator structure. In some embodiments, the respective computing nodes 320 may include a communication circuit configured to provide communication among the plurality of computing nodes via one or more interconnection busses. In some examples, an interconnection bus may be a wireless communication bus, generally indicated by reference numeral 340.

In some embodiments, the respective computing nodes 320 may be configured as a computing node in a Bluetooth™ mesh network architecture. One or more of the respective computing nodes 320 relay data signals among computing nodes 320 associated with the textile computing platform.

In scenarios where a pair of computing nodes 320 may be physically positioned apart and may not have a direct interconnection bus therewith, the pair of computing nodes 320 may share data signals via other computing nodes 320 acting as signal relays in a mesh network.

In scenarios where some computing nodes 320 of the textile computing system 300 may have a wired interconnection bus and where some other computing nodes 320 of the textile computing system 300 may have a wireless interconnection path, the textile computing system 300 may be configured to interconnect the plurality of computing nodes 320 distributed about a textile 310, even when some computing nodes 320 may not have a common interconnection bus medium (e.g., wired interconnection bus versus wireless interconnection bus).

In some embodiments, one or more of the computing nodes 320 may be interconnected with other computing nodes 320 based on a combination of wired interconnection busses and wireless interconnection busses. In scenarios where computing nodes 320 may be physically distant or may be separated by numerous intermediary computing nodes 320, the plurality of computing nodes 320 may conduct operations to determine a most efficient path for data transmission among the plurality of computing nodes 320. For example, a data transmission path between a first computing node and a second computing node may be based at least in part on: (i) a wireless interconnection bus (e.g., via a Bluetooth™ mesh-based network); and (ii) a wired interconnection bus (e.g., conductive fiber network integral to the textile 310).

In some embodiments, the respective nodal regions 330 of the textile 310 may include a socket or receptacle for removably receiving a computing node therein, such that off-the-shelf computing nodes may be received therein. In scenarios where a wireless communication bus is implemented, one or more of the plurality of computing nodes may be configured to conduct operations for peer-to-peer discovery of other computing nodes. Operations for peer-to-peer discovery of proximal computing nodes may be beneficial in scenarios where a user of the textile computing platform 300 may customize the type of computing nodes to be installed at the textile 310.

In some embodiments, the interconnection bus may be a wired interconnection bus based on a conductive fiber network of the textile 310 integrated therein. For ease of exposition, in FIG. 3, a two-wire communication bus 342 is illustrated for providing a bi-directional bus to transmit/receive data or power signals among the plurality of computing nodes 320.

In some embodiments, the respective computing nodes 320 may include a dedicated power source, thereby reducing the need to transmit power signals along the interconnection bus. In some other embodiments, one or more of the plurality of respective computing nodes 320 may be configured without a power source. In the latter embodiment, the computing node being configured without a power source may receive power signals via the wired interconnection bus or via inductive charging power structures integrated in the textile 310.

As described, the textile 310 illustrated in FIG. 3 may be a sock worn by a user. In some embodiments, it may be beneficial to collaboratively retrieve sensory data based on the plurality of nodal regions 330 for detecting changes in modality of the sock (or other garment). As a non-limiting example, modalities of the sock may be based on detected environmental conditions proximal to the plurality of nodal regions 330. For example, one or more of the computing nodes 320 may collectively determine that the sock may be in a moist environment, at least because sensory data retrieved by at least two or more of the computing nodes 320 may indicate moisture levels beyond a threshold value. In some scenarios, the one or more computing nodes 320 may collectively determine that the sock may be in a “cool” environment at least because sensory data retrieved by at least two or more of the computing nodes 320 may indicate temperature readings beyond a threshold value.

In some embodiments, the one or more computing nodes 320 may collectively determine modalities based on sensory data sets transmitted among the plurality of computing nodes 320, via the interconnection bus (330 or 332). In response to identifying one or more changing modalities, in some embodiments, the plurality of computing nodes 320 may respectively be configured to provide actuator output via one or more actuator structures as a combined whole to a user of the textile 310. For example, in scenarios where the computing nodes 320 determines that the sock textile 310 may be in a “cooler” environment, the plurality of computing nodes 320 may respectively be configured to provide actuation signals for providing heating to the textile 310.

Continuing with the present example, the required magnitude or degree of heating provided to the sock textile 310 by the plurality of computing nodes 320 may vary from computing node to computing node. For example, it may be beneficial to provide a greater degree of heating to nodal regions proximal to portions of the sock where a user's toes may be located and to provide relatively lesser degree of heating to nodal regions proximal to portions of the sock where a user's angle may be located. Accordingly, the plurality of computing nodes 320 may be configured to collectively determine a modality of the textile 310 and, in response, collectively coordinate actuation of actuator structures.

As another non-limiting example, the plurality of computing nodes 320 may be configured to collectively determine a modality of the textile 310 and, in response, collectively coordinate actuation of mechanical actuator structures to provide vibratory sensation to the user's foot over time. For instance, the vibratory sensation to the user's foot may be a rippling vibration sensation that begins near the user's toes and progresses towards the user's ankle. In some examples, mechanical actuator structures may be configured to provide notification signals to the textile user or may be configured to provide physical physiotherapeutic relief, akin to massage therapy. Other types of actuator structure output may be contemplated.

In examples described herein, the textile computing platforms may include a plurality of computing nodes respectively including a node processor for conducting operations substantially independently of other computing nodes. In some other embodiments, it may be beneficial to provide at least one computing node designated as a central computing node configured to provide centralized control operations.

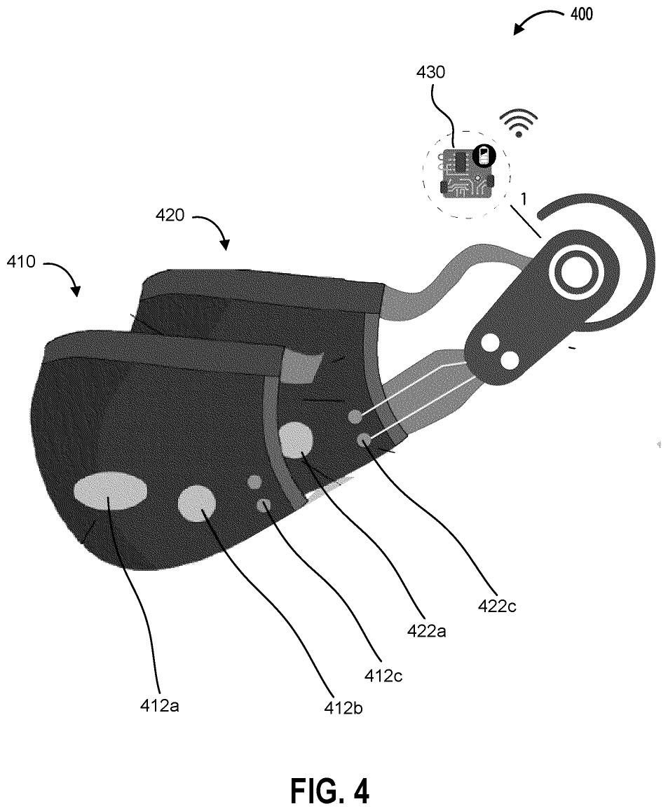

Reference is made to FIG. 4, which illustrates a textile computing platform 400, in accordance with another embodiment of the present disclosure. In FIG. 4, the textile computing platform 400 may be a facial garment, such as a mask among other examples, adapted to be worn by a user. The facial garment may be worn over the mouth or the nose of a user to protect the user's respiratory system. Other types of garments having similar features described herein may be contemplated.

The textile computing platform 400 may include features such that textiles may be modularly added or removed. For example, the textile computing platform 400 illustrated in FIG. 4 may include a first mask portion 410 and a second mask portion 420. The first mask portion 410 may be associated with an outer layer of the facial garment when worn by a user. The second mask portion 410 may be associated with an inner layer of the facial garment when worn by the user.

The respective first mask portion 410 and the second mask portion 420 may include a textile including a plurality of nodal regions. The mask portions may include a plurality of computing nodes respectively coupled to a nodal region of the textile. Each of the computing nodes may include at least a node processor and at least one of an actuator structure or a sensor structure coupled to the node processor.

For example, the first mask portion 410 may include a first computing node 412a having a heating structure (e.g., a resistive coil) integrated thereon. The first mask portion 410 may include a second computing node 412b having a chemical sensor, such as a volatile organic compound sensor, integrated thereon. Further computing nodes coupled to corresponding nodal regions of the first mask portion 410 may be contemplated.

The second mask portion 420 may include one or more computing nodes coupled to corresponding nodal regions of the second mask portion 420, including a third computing node 422a having a pressure or fiber stretch sensor. Other computing nodes may be coupled to the second mask portion 420 at other nodal regions.

Further, the first mask portion 410 may include a textile interconnection structure 412c for providing an electrical interconnection bus with a corresponding textile interconnection structure 422c of the second mask portion 420. The electrical interconnection bus may be configured to provide a coupling mechanism to convey data or power signals between computing nodes of the first mask portion 410 and the second mask portion 420.

The second mask portion 420 may include one or more computing nodes coupled to corresponding nodal regions of the second mask portion 420, including a fourth computing node 422a.

In some embodiments, the textile computing platform 400 illustrated in FIG. 4 may further include a supplemental computing node 430 adapted to be interconnected with the textile of the first mask portion 410 and the second mask portion 420. The supplemental computing node 430 may be constructed at least in part based on textiles having sensors associated with detecting PPG, ECG, or temperature signals, among other examples, integrated therein.

The textile computing platform 400 may be configured such that respective computing nodes associated with a nodal region of textile may be in communication with at least one other computing node via an interconnecting bus, such that sensory data or actuator signals may be transmitted among the respective computing nodes.

To illustrate operations of the textile computing platform 400, an example associated with the facial garment may be provided. In some examples, the respective computing nodes of the plurality may conduct operations to detect sensory data associated with the corresponding nodal region of the textile (e.g., first mask portion 410 or second mask portion 420). A computing node configured with a volatile organic compound sensor and an adjacent computing node configured with a temperature sensor may substantially independently generate sensory data associated with the user of the facial garment.

Overtime, sensory data may be transmitted via interconnection busses from respective computing nodes to other computing nodes. In some examples, interconnection busses may include conductive fibers associated with the facial garment textile. In other examples, interconnection busses may include wireless communication channels based on Bluetooth™ communication or near-field communication protocols, among other examples. As sensory data generated by the respective computing nodes may be transmitted to other computing nodes, in some embodiments, the plurality of computing nodes may be configured to determine a modality or change in modality of the textile(s) associated with the textile computing platform 400.

For example, a change in modality may include a change in temperature beyond a threshold value associated with a user wearing the facial garment, or a change in detected concentration of volatile organic compounds beyond a threshold value within the cavity between the facial garment and the user.

Based on the detected sensory data, one or more of the computing nodes may transmit to the plurality of computing nodes one or more actuator signals, such that the respective computing nodes may activate actuating structures, and such that the plurality of computing nodes may collectively activate actuating structures in concert to provide actuating output to the user.

As non-limiting examples, in response to detecting decreasing environmental temperatures at an exterior surface of the facial garment, the plurality of computing nodes may be configured to generate actuating signals to selectively generate heat via resistive textile fibers at nodal regions of the textile associated with nasal-oral regions of the user. In another example, in response to detecting increasing strain or stretch among textile fibers of the facial garment, the plurality of computing nodes may be configured to generate actuating signals to generate haptic or vibratory feedback collectively across the plurality of nodal regions of the textile, thereby providing the user with indications that the fit of the mask may be non-optimal.

Other practical applications of retrieving sensory data at a plurality of computing nodes and, subsequently, transmitting actuating signals to the plurality of computing nodes to collectively provide actuating feedback in concert to a textile user may be contemplated.

Embodiments of textile computing systems disclosed herein may be implemented such that two or more textiles (e.g., textile garments) may be interconnected for data or power transmission. In some embodiments, a textile computing system may include a plurality of textiles. The respective textiles may include a plurality of nodal regions associated with computing nodes. The plurality of computing nodes among the plurality of textiles may be interconnected based at least in part on: (i) a wireless interconnection bus (e.g., via a Bluetooth™ mesh-based network, among other mesh-based network examples); and (ii) a wired interconnection bus (e.g., conductive fiber network integrated in respective textiles). Such embodiments of textile computing systems may be beneficial for providing dynamic expansion of a textile computing system based on modular peer-to-peer discovery of textiles having computing nodes thereon. Further, such embodiments of textile computing systems may be beneficial for allowing varying textile configurations to be interconnected or combined for providing distributed sensory data acquisition or distributed actuating output to a user of textile computing systems.

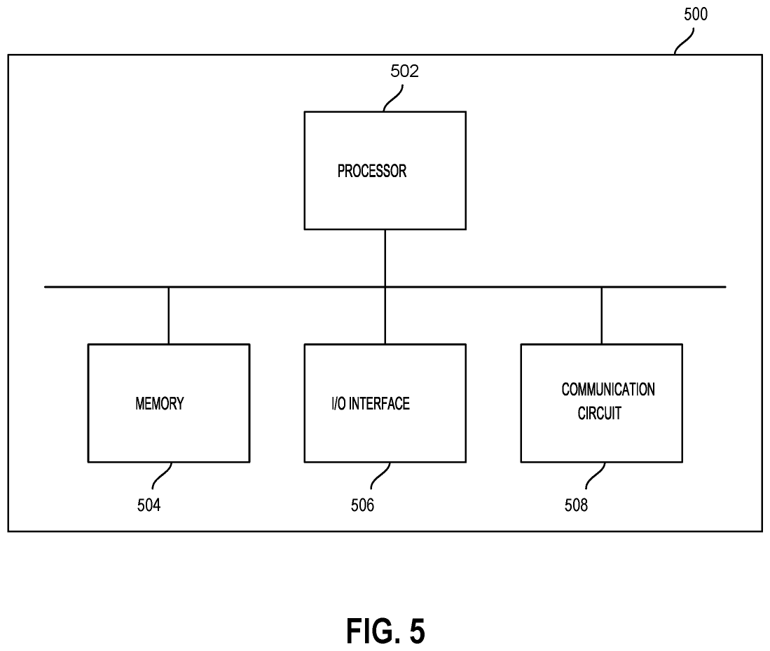

Reference is made to FIG. 5, which illustrates a block diagram of a computing device 500, in accordance with an embodiment of the present disclosure. As an example, one or more of the computing nodes of FIG. 1 may be implemented using the example computing device 500 of FIG. 5.

The computing device 500 may include at least one processor 502, memory 504, at least one I/O interface 506, and at least one communication circuit 508.

The processor 502 may be a microprocessor or microcontroller, a digital signal processing processor, an integrated circuit, a field programmable gate array, a reconfigurable processor, a programmable read-only memory, among other examples.

The memory 504 may include a computer memory that may be located either internally or externally such as, for example, random-access memory, read-only memory, compact disc read-only memory, electro-optical memory, magneto-optical memory, erasable programmable read-only memory, and electrically-erasable programmable read-only memory, Ferroelectric RAM.

The I/O interface 506 may enable the computing device 500 to interconnect with one or more input devices, such as a keyboard, mouse, camera, touch screen and a microphone, or with one or more output devices such as a display screen and a speaker.

The communication circuit 508 may be configured to receive and transmit data sets, for example, to a target data storage or data structures.

Systems described in the present disclosure may provide embodiments of garment systems. A garment system may include a plurality of garments, and the respective garments may include one or more computing nodes positioned and configured to interface with an anatomical portion of a user. In some embodiments, the computing nodes may include one or more sensors or actuators for interfacing with the user's skin. Other types of interactions with a user's physiological features may be used. Further, types of interfaces may include vibrational, optical, or audible interfaces, among other examples.

In some embodiments, the computing nodes may include one or more sensors for detecting bio-potential signals associated with the user. For example, the one or more sensors may include electrodes for interfacing with the user's skin to record electromyography (EMG), electrocardiography (ECG), electroencephalography (EEG), and/or electrooculography (EGO) data.

In some embodiments, the computing nodes may include one or more actuators for providing output to the user's skin. In some embodiments, the one or more actuators may provide thermal (e.g., heating or cooling) output, haptic or tactile output, electrical stimulation, or other types of output to the user.

In some embodiments, the respective garments may include one or more docks for removably receiving computing nodes. Such example docks may allow a user to dynamically add, remove, or configure types of computing nodes for a textile garment based on environmental conditions or anticipated user activity.

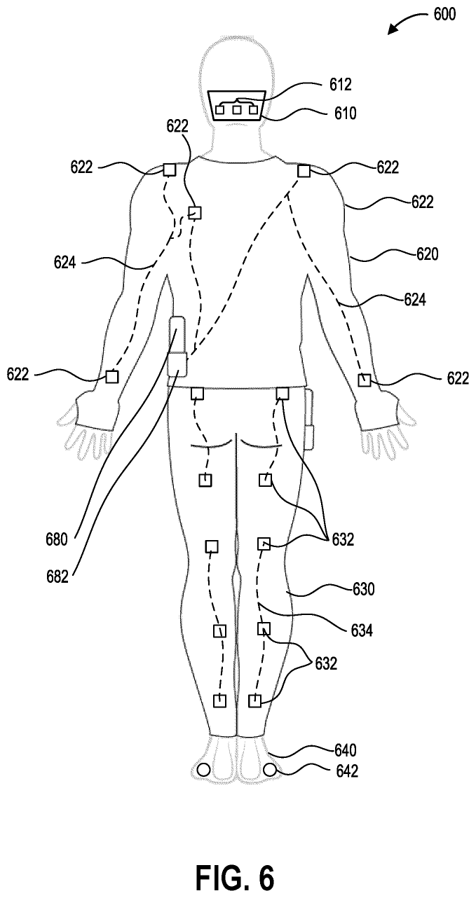

Reference is made to FIG. 6, which illustrates a garment system or textile computing system 600, in accordance with embodiments of the present disclosure. In FIG. 6, the garment system 600 may include a facial garment 610 (e.g., personal protective mask, balaclava, or the like), a shirt 620, pants 630, or a pair of socks 640.

The garment system 600 may include a plurality of embodiments of the computing nodes described in the present disclosure. For example, the facial garment 610 may include one or more computing nodes 612. The computing nodes 612 may interconnect with other computing nodes via physical interconnections (e.g., electrical conductive fibers of the textile) or via wireless communication methods.

The shirt 620 may include one or more computing nodes 622 positioned to interface with various anatomical features of the user. For example, the computing nodes 622 may be positioned at the yoke of the shirt 620 for interfacing with an upper body or shoulder region of the user. Other computing nodes 622 may be positioned along the arms or across the chest of the user for interfacing with arms, torso, or chest regions of the user. The computing nodes 622 may interconnect with other computing nodes via physical interconnections 624 or via wireless communication protocols or mediums.

The pants 630 may include one or more computing nodes 632 interfacing with different portions of the user's lower body, such as the waist region, the thigh region, muscles surrounding the user's knees, calves, angles, or other anatomical features of the user. The plurality of computing nodes 632 may interface with other computing nodes of the pants 630 based on physical interconnections 632 or via wireless communication methods as described in the present disclosure.

The socks 640 may include one or more computing nodes 642 for interfacing with one or more portions of the user's feet, such as the heel, the arch of the user, the region adjacent the toes, among other example portions of the user's feet.

The described example computing nodes illustrated in FIG. 6 may include one or more features of computing nodes described in the present disclosure.

The garment system 600 may be dynamically configured to include additional or fewer garments at a particular point in time. In some scenarios, the user may add or remove layers of garments based on environmental conditions or based on the type of anticipated activity the user may be undertaking. For example, where the user may be preparing to go for a run on a relatively cold day, the user may add a hat, a balaclava (e.g., facial garment to protect the face), a long sleeve shirt, a pair of athletic pants, or a pair of socks. In other examples, where environment conditions may include rising ambient temperatures, the user may substitute the long sleeve shirt for a t-shirt and may remove the facial garment. Other configurations may be contemplated.

In some embodiments, as the garment system 600 is dynamically configured, the plurality of computing nodes may conduct operations for detecting or discovering adjacent or proximally located nodes across the disparate garments of the garment system 600. In the scenario where the user removes the long sleeve shirt, one or more computing nodes of the garment system 600 may identify that the computing nodes 622 of the shirt 620 may no longer be in communication, and may conduct operations to cease communications or receiving sensory data from computing nodes 622 of the shirt 620.

In the scenario where the user substitutes the long sleeve shirt with a t-shirt having one or more computing nodes positioned at disparate locations of the t-shirt, the one or more computing nodes of the garment system 600 may identify the recently detected computing nodes associated with the t-shirt, and may conduct operations to conduct communications with the recently detected computing nodes over time.

In some scenarios, anatomical features of a user may be subject to repeated motions (e.g., behind the user's knees during the user's gait). To minimize occurrences of a physical communication interconnection (e.g., fiber) wearing out due to repetitive motion or other potential signal degradation characteristics associated with repetitive motion, in some embodiments, it may be beneficial to interconnect computing devices across anatomical features experiencing repetitive motions using wireless communication interconnections.

In some embodiments, wireless communication among the plurality of computing nodes may be conducted based on wireless communication operations, such as Bluetooth™ Low Energy, among other examples of wireless communication protocols. In some embodiments, wireless communication operations may be conducted among computing nodes positioned on a given garment. Referring again to FIG. 6, in some embodiments, a subset of computing nodes 632 may be interconnected based on a physical communication medium (e.g., electrically conductive fiber in the garment textile). In some example garments, a subset of computing nodes 632 may communicate with other computing nodes 632 via wireless communications operations (e.g., BLE). For example, in FIG. 6, the subset of computing nodes 632 proximal the upper-leg region may wirelessly communicate with another subset of computing nodes 632 proximal the lower-leg region. In another example, near-field communication protocols may be used where there may be overlap among adjacent textile garments (e.g., shirt and pant in a region where the shirt overlaps/covers the pants). Other configurations of communication interconnections may be contemplated.

Based on embodiments described in the present disclosure, garment systems may be dynamically configured as textile computing systems having a plurality of positioned computing nodes for detecting and generating bio-potential data associated with a user. The bio-potential data may include data corresponding to EMG, ECG, EEG, or EOG based on sensory information detected at a plurality of disparate anatomical locations about the user's body. In some embodiments, the computing nodes may generate, store, or transmit to other computing nodes other types of physiological data, such as inertial measurement unit (IMU) data, temperature, heat flux, among other examples.

In some embodiments, the plurality of computing nodes may include operations for relaying sensory data to a gateway device 680 (FIG. 6). The gateway device 680 may be a mobile computing device, among other examples, for combining data sets received from the plurality of computing nodes of the textile garment system 600. In some embodiments, the gateway device 680 may be a data router device positioned within a user environment, and the gateway device 680 may transmit messages to and receive messages from computing nodes of example textile computing platforms described in the present disclosure.

In some embodiments, the gateway device 680 may be received within an interconnection pocket 682 and may be configured to electrically interconnect with the plurality of computing nodes of the garment system 600. In some embodiments, the gateway device 680 may include a processor and a memory having processor-executable instructions for combining sensory and other bio-potential data, and operations for processing the data sets for providing user activity and/or health status analytical insights. In some embodiments, the gateway device 680 may be configured to transmit combined datasets to an external or off-site computing device (e.g., cloud computing server) for processing or storage.

In some embodiments, the garment system 600 may be configured to collaboratively detect or generate bio-potential or sensory data from the plurality of computing nodes (e.g., associated with nodal regions positioned across the user's anatomical features). In some embodiments, the garment system 600 may be configured to conduct operations for detecting changes in user modality. For example, the bio-potential or sensory data sets, in combination, across the user's anatomical features may be used for detecting user movement. In some embodiments, the bio-potential or sensory data sets, in combination, across the user's anatomical features may be used for generating health evaluation, heath metrics, and/or medical diagnosis operations/analysis.

To illustrate, the garment system 600 may be configured to conduct operations of gait analysis, biofeedback training, or real-time coaching, among other examples, for the user. In some scenarios, such bio-mechanical or physiological analysis operations conducted in substantially real-time may be based on precise alignment of timestamps associated with sensory or bio-potential data sets to provide the bio-mechanical or physiological analysis for the user.

If sensory data associated with computing nodes detecting muscle movement proximal to the user's knees is associated with a timestamp that may be offset from sensory data associated with computing nodes positioned at the arch of the user's foot, example gait analysis may not be precise. It may be beneficial to provide systems and methods of aligning timestamps or synchronizing sensory or bio-potential data records over time, such that higher-level user analysis may be conducted.

In some embodiments, the plurality of computing nodes among the garments of the garment system 600 may latch onto or obtain a trigger from a real-time clock signal, such that sensory or bio-potential data generated and retrieved from the respective computing nodes may be associated with aligned time stamps over time.

For example, when a user takes a step, the plurality of computing nodes may generate sensory data associated with anatomical features proximal to the computing node placement (e.g., computing node at arch of user foot, computing node at ball of user foot, computing node at calf of users leg, computing node behind knees of user's leg, among other examples). It may be beneficial for operations of gait analysis to combine sensory data having time stamps at a moment that the user's foot makes contact with the ground, and at a plurality of other times as the user progresses through a walking motion.

At each of the respective times during a user's gait, accuracy of the gait analysis may only be as accurate as alignment of the sensory data generated by the plurality of computing nodes. For example, the plurality of sensory data points associated with when the user's foot makes contact with the ground may be a set of sensory data points representing the plurality of anatomical positions of the user, and precise timestamp alignment of such sensory data points may be important to providing accurate user gait analysis. Thus, in some embodiments, the garment system 600 may include one or more processors (e.g., processor of gateway device 680 or one or more other processors of the computing nodes) for generating a synchronous clock from which the plurality of one or more processors may rely on for aligning sensory data sets.

In some embodiments, the gateway device 680 may transmit a real-time clock signal to a plurality of nodes, such that the plurality of nodes may synchronize their data record timestamps. In some embodiments, the real-time clock signal may be updated on a periodic basis (e.g., every hour, 2 hours, . . . 12 hours, or other time period).

In some embodiments, the gateway device and/or the respective computing nodes may conduct operations to determine timing delays in communication transmissions among computing nodes and the gateway device. The timing delays may be determined based on device handshaking processes, and may include operations of a round-trip-time calculation. Other examples of timestamp synchronization or alignment may be used.

Embodiments of the present disclosure include a textile computing system configured to dynamically enumerate a set of computing nodes of respective textile garments. The set of computing nodes may be a subset of a universal set of computing nodes compatible for being configured with the textile computing system.

In some embodiments, respective textile garments may include head garments (e.g., hats, headbands, among examples), facial garments (e.g., masks, balaclavas, among examples), upper-body garments (e.g., long sleeve shirts, undergarments, short sleeved shirts, among examples), lower-body garments (e.g., pants, shorts, among examples), footwear (e.g., socks, stockings, shoes, among examples), or other garments configured to be positioned over or proximal to anatomical features of a user.

Textile garments may include nodal regions having computing nodes, and the computing nodes may include sensory-actuating devices positioned within the nodal regions. In some embodiments, sensory-actuating devices may include specific types of sensors for detecting particular physiological characteristics, such as electrical characteristics across the skin surface of a user, blood pressure, movement (e.g., via inertial measurement units), or temperature, among other examples.

In some scenarios, a user may add or remove layers of textile garments based on environment conditions (e.g., heat or cold conditions) or based on the type of anticipated activity the user may be undertaking (e.g., running during cold weather versus swimming). Embodiments of the textile computing systems may be configured to dynamically discover or enumerate a network of computing nodes, such that operations for physiological analytics may be identified based on analytical capability of sensory data records of the enumerated computing nodes. The enumerated network of computing nodes may represent a current textile system state.

To illustrate, when a user is conducting warm-up exercises, the user may be wearing a hat, a long-sleeve shirt having computing nodes positioned to interface with the user's arms, chest, shoulders, or other anatomical features, or a pair of long pants having computing nodes positioned to interface with skin surfaces adjacent a plurality of leg muscle types. During warm-up exercises, the textile computing system may be configured to discover the plurality of computing nodes associated with the plurality of textile garments, and may be configured to generate physiological analytics based on a series of sensory data records received from the enumerated subset of computing nodes. As a non-limiting example, the current textile system state may be configured to generate bio-mechanical feedback to assist the user with reaching a desired muscle warm up state (e.g., based on heart rate, muscle movement cycles, among other factors).

At a subsequent point in time, the user may change into a pair of shorts and may add socks or shoes, respectively including computing nodes at nodal regions positioned about the textile garments. The addition or removal of one or more textile garments may provide a subsequent textile system state.

The textile computing system may further be configured to discover that textile garments have been added or removed, resulting in a subsequent combination of computing nodes configured for physiological analytics. For example, the textile computing system may be configured to discover another plurality of computing nodes associated with the subsequent textile system state for generating physiological analytics.

In some embodiments, the textile computing system may be configured to conduct operations for dynamically identifying a current textile system state, determine sensory actuating devices associated with computing nodes presently positioned about anatomical features of the user, and conduct operations to provide physiological analytics based on a series of sensory data records received from the identified or enumerated subset of computing nodes. The physiological analytics may be configured to provide physiological insights based on sensory data records collected among different anatomical portions of the user's body over a spectrum of time.

In some embodiments, the textile computing system may be configured to generate and transmit actuating signals to the one or more enumerated subset of computing nodes based on the physiological analytics for providing feedback to a user on a substantially real-time basis. For example, the actuating signals may provide one or a combination of haptic feedback, thermal feedback, or acoustic feedback, or electrical stimulation, among other types of feedback, for guiding the user during the user's activities.

As an example, the physiological analytics may include user gait analysis. In some embodiments, the textile computing system may conduct operations for determining whether the enumerated subset of computing nodes include a defined quantity or set of sensory-actuating devices positioned at desirable portions of anatomical features of the user's body to retrieve sufficient time-series data for sufficiently providing the user gait analysis.

In response to determining that the enumerated subset of computing nodes as a combination may be acceptable (e.g., in quantity or data type) for generating a plurality of time-series sensor data sets for conducting gait analysis, the textile computing system may be configured to generate actuating signals to the one or more enumerated subset of computing nodes based on physiological analytics for generating feedback to a user on a substantially real-time basis. For example, the actuating signals may be transmitted to configure the enumerated subset of computing nodes to provide haptic feedback to targeted portions of the user's anatomical features as feedback for improving the user's gait or movement form. Other examples of physiological analytics and associated actuating signal generation may be used.

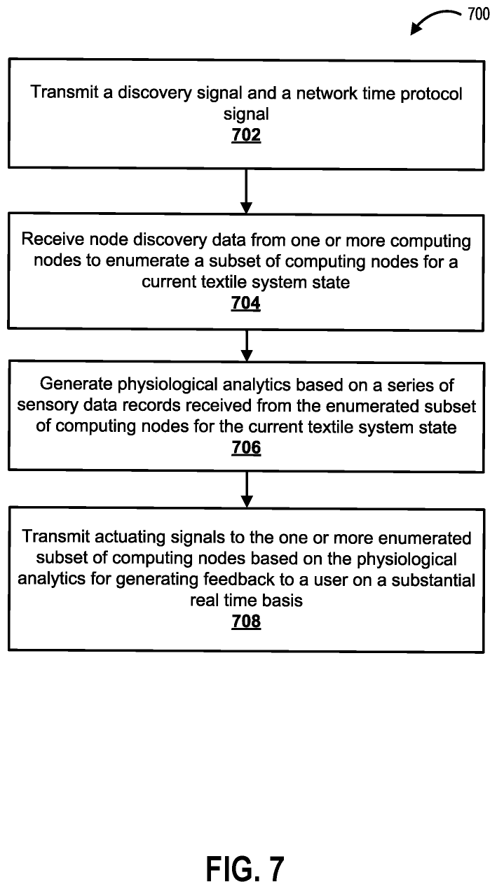

Reference is made to FIG. 7, which illustrates a method 700 of a textile computing system, in accordance with embodiments of the present disclosure. The method 700 may be conducted by a gateway processor of a gateway device 680 of the textile garment system 600 (FIG. 6) or textile computing platform. Processor-executable instructions may include operations such as data retrievals, data manipulations, data analysis, data storage, or other operations, and may include computer-executable operations.

To illustrate features of the method 700, the operations will be described as being executed by a gateway processor of the gateway device 680. The gateway device 680 may be a mobile computing device configured to provide centralized data set aggregation and physiological analytics generation based on a plurality of enumerated computing nodes. The plurality of enumerated computing nodes may be based on a combination of disparate textile garments currently worn by a user for defining a current textile system state.

The textile computing platform may include one or more textiles, and the respective textiles may include a plurality of nodal regions for interfacing with one or more anatomical features of a user. The respective nodal regions may be configured to be in communication with at least one nearby nodal region.

The textile computing platform may include at least one computing node positioned substantially within one of the textile nodal regions. The respective computing nodes may include one or more sensory-actuating devices. Sensory-actuating devices may be a combination of a sensor device and an actuating device for generating a physical output to a user.

In some embodiments, computing nodes may respectively be positioned at positions across anatomical features of a user for a defined physiological analytics types. For example, when a shirt garment is worn by a user, a particular computing node may be positioned at a position on a long sleeve shirt garment that may interface with a chest region of the user. In another example, particular computing nodes may be positioned across at least one of a foot of the user, a position proximal to a knee of the user, or a position proximal upper leg or lower leg muscles of the user for generating gait analysis for the user.

The gateway device 680 may be in communication with the plurality of computing nodes via a textile communication network. The textile communication network may include a combination of conductive fibers integral to respective textiles and wireless communication circuits for wireless communication. In some scenarios, a physical conductive transmission path between a long-sleeve shirt and a pair of shorts may not be feasible. In some embodiments, a textile communication path across disparate textiles (e.g., from socks to pants, pants to a shirt, shirt to a facial garment, among other communication paths) may be based on wireless communication protocols.

At operation 702, the gateway processor may transmit, via the textile communication network, a discovery signal and a network time protocol signal. In some embodiments, the network time protocol signal may be configured for synchronizing a plurality of computing nodes, such that sensory data records generated by the respective computing nodes may be associated with time-stamps to associate a plurality of sensory data generated by the plurality of computing nodes at successive points in time.

In some embodiments, the discovery signal may include operations for sending ping messages, and the gateway processor may subsequently determine availability of computing nodes based on receiving acknowledge messages from respective computing nodes.

In some embodiments, the gateway device may conduct operations to detect whether particular textile garments are worn, thereby allowing the textile computing system to determine which textile garment should be considered during operations of physiological analysis.

In some embodiments, the user's body may be a common medium as a basis for operations to determine presence of garments on the user's body. For example, the gateway device or computing nodes may conduct operations to determine impedance measurements for identifying whether a textile garment is being worn by a user. In some other embodiments, operations for determining temperature or heat flux may be identified for determining whether a particular textile garment is being worn by a user (e.g., when a textile garment is worn by a user, the textile garment may experience body heat from the user).

In some embodiments, the gateway device or computing nodes may conduct operations associated with proximity sensing. For example, near-field communication operations may be configured to identify whether one garment may be adjacent to and near (e.g., within a few centimeters or inches) of another garment.

In some embodiments, the gateway device or computing nodes may conduct operations associated with identifying continuity among a plurality of textile garments based on a continuous path created based on snap button connectors (or similar devices) (e.g., shirt to pants, pants to socks) for providing a wired communication path among textile garments, thereby providing a confirmation that garments are being worn by the user.

At operation 704, the gateway processor may receive node discovery data from one or more computing nodes to enumerate a subset of computing nodes for a current textile system state. In some embodiments, the node discovery data may include acknowledge messages from computing nodes that are available for generating sensory data records for the current textile system state.

In some embodiments, the node discovery data may include messages recording a sensory device type, thereby identifying a physiological characteristic that may be recorded by the sensory device. For example, the sensory device type may indicate that the sensory device detects temperature or heat flux across the user's skin, moisture level across the user's skin, movement of muscles proximal to the sensory device at the position on the user's skin, or other types of sensory data type.

In some embodiments, the discovery data may include location data of the sensory device about the user's anatomical features. In an example of a sock, the discovery data may include data messages indicating that the computing node is positioned proximal to an arch of the user's foot or proximal to the user's ankle. Other data messages or data types may be provided in the discovery data.

In some embodiments, the gateway processor may conduct operations to enumerate the subset of computing nodes identified by the discovery data for identifying a current textile system state. For example, the gateway processor may determine that the current textile system state includes a head garment (including electrodes for providing data sets for electroencephalography, among other types of data), a t-shirt garment (including sensors for detecting heart rate or heart characteristics over time), a pair of pants (including sensors with inertial movement units for detecting user muscle movement), or socks (including sensors for detecting muscle movement or pressure). Other types of textile garments may be contemplated.

In some scenarios, the enumerated subset of computing nodes may be greater in number or less in number depending on the combination of textile garments being worn by the user. Accordingly, embodiments of the textile computing platform may dynamically enumerate a textile system state as a prelude to generating physiological analytics.

In scenarios where sensory data sets may not be optimal for generating a particular type of physiological analysis, the textile computing platform may decline to conduct those physiological analysis operations. For example, in scenarios where a user may not be wearing a pair of socks having computing nodes for detecting pressure or foot movement, or may not be wearing a knee brace having computing nodes for detecting knee/joint movement, the textile computing platform may determine that there is insufficient sensory data for providing a useful gait analysis.

In another example, a user may not be wearing textile garments for detecting heart rate. Accordingly, the textile computing platform may determine that there is insufficient sensory data for providing a useful heart rate efficiency analysis during the user's physical activities.

At operation 706, the gateway processor may generate physiological analytics based on a series of sensory data records received from the enumerated subset of computing nodes for the current textile system state. The respective sensory data records may be associated with a time-stamp based on the network time protocol signal.

In some embodiments, the series of sensory data records may include at least one of inertial measurement unit data, temperature or heat flux data at a skin surface of a user, or electrical characteristics across a skin surface of the user.

As described herein, in some embodiments, the textile communication network may include, at least in part, wireless communication networks (e.g., Bluetooth™ Low Energy or a combination of Bluetooth™ Low Energy and near-field communication protocol operations). It may be challenging to receive sensory data records in substantial real time at the gateway device 680, at least due to potential data transmission delays across the wireless communication networks.

To address delays in sensory data record transmission, in some embodiments, the respective sensory data records may be associated with a time-stamp based on one or more network time protocol signals. The network time protocol signals may be reference clock signals, such that sensory data records received from the plurality of enumerated computing nodes at a particular point-in-time may be correlated with one another.

The gateway processor may generate physiological analytics based on the correlated sensory data records at particular points-in-time for the current textile system state

At operation 708, the processor may transmit actuating signals to the one or more enumerated subset of computing nodes based on the physiological analytics for generating feedback to a user on a substantially real time basis.

In some embodiments, the actuating signals may be a group of actuating signals that are selectively transmitted to target or identified computing nodes, and the actuating signals may in combination be configured to cause the respective enumerated computing nodes to provide feedback at anatomical features of the user's body. As an illustrating example, the actuating signals may cause a plurality of enumerated computing nodes to provide, in concert, haptic feedback by computing nodes proximal to the user's knees. Thus, the enumerated computing nodes may provide haptic feedback as a unit.

In some embodiments, a subset of the enumerated computing nodes may be configured to provide other types of concurrent feedback to the user at other anatomical features of the user's body. For example, in concert with providing haptic feedback by the computing nodes proximal to the user's knees, the enumerated computing nodes may provide thermal feedback to the user's calves (via computing nodes positioned in pant legs) or the user's foot (via computing nodes positioned in socks). Thus, the enumerated subset of computing nodes may include one or more actuating devices, and the transmitted actuating signals may be configured to generate a combination output at the one or more actuating devices.

In some embodiments, the gateway processor may conduct operations to determine a subset of physiological analytics that correspond to at least one of the identified sensor device type or a sensor position. The subset of physiological analytics may be based on capabilities of sensory data records from the identified sensor device type or sensor position of discovery data.

To illustrate, the gateway processor may determine that gait analysis may not be possible in the event that enumerated computing nodes do not include computing nodes positioned proximal to the user's foot, knees, or other anatomical features of the user's body desirable for generating gait analysis.

In another example, the gateway processor may determine that blood pressure or heart rate analysis may be possible in the event that enumerated computing nodes include computing nodes (e.g., of a long-sleeve shirt) may be positioned about the user's chest, wrists, or other anatomical features of the user's body desirable for detecting physiological data sets pertinent to generating blood pressure or heart rate analysis.

In some embodiments, physiological analytics may include one of gait analysis, biofeedback training, auto-coaching operations in substantial real-time, or gamification operations. Other types of physiological analytics may be contemplated.

In some embodiments, the respective computing nodes of the textile computing system may include a node processor, at least one sensory-actuating device coupled to the node processor, and a node memory coupled to the processor. The node memory may store processor-executable instructions that, when executed by the node processor, configure the node processor to conduct operations such as generating sensory data records based on the at least one sensory-actuating device and associating a time-stamp based on a received network time protocol signal.

In some embodiments, the respective node processors may receive one or more actuating signals for generating sensory-actuating output for application across an anatomical feature of the user as a combination output of the enumerated subset of computing nodes.

In some embodiments, the at least one sensory-actuating device may be configured to provide at least one of haptic output, heating or cooling output, or acoustic output across the anatomical feature of the user.

In some embodiments, the node processor may be configured to generate physiological analytic portions based on the sensory data records generated at the present computing node. The physiological analytic portions being subset analytics for generating physiological analytics by the gateway processor based on a combination of computing nodes of the textile computing system.

In some embodiments, the textile computing system may include features of a distributed computing system that includes computing nodes for conducting operations of analytics portions. The gateway processor may receive physiological analytics portions data from the plurality of computing nodes for generating an overall, larger picture physiological analytics.

In some embodiments, respective computing nodes may process raw sensor data, and may determine metrics from the raw sensor data on a substantially real-time basis. In some embodiments, the respective computing nodes may transmit the determined metrics (e.g., generated by the computing node) to the gateway device for further or “big-picture” analysis based on metrics data received from the plurality of computing nodes.

In some embodiments, the respective computing nodes may be configured to determine validity and/or quality of detected data signals, and transmit data signals to the gateway device in the event that the detected data signals meet a threshold metric of validity and/or quality threshold or criterion.

In some embodiments, the respective computing nodes may be configured to identify specific patters among recorded raw data sets indicating an event or an alteration in trends. In such example scenarios, the computing nodes may transmit such recorded raw data sets to the gateway device for subsequent and/or detailed data analysis.

The term “connected” or “coupled to” may include both direct coupling (in which two elements that are coupled to each other contact each other) and indirect coupling (in which at least one additional element is located between the two elements).

Although the embodiments have been described in detail, it should be understood that various changes, substitutions and alterations can be made herein without departing from the scope. Moreover, the scope of the present disclosure is not intended to be limited to the particular embodiments of the process, machine, manufacture, composition of matter, means, methods and steps described in the specification.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

The description provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

The embodiments described herein are implemented by physical computer hardware, including computing devices, servers, receivers, transmitters, processors, memory, displays, and networks. The embodiments described herein provide useful physical machines and particularly configured computer hardware arrangements.

As can be understood, the examples described above and illustrated are intended to be exemplary only.

What is claimed is:

1. A textile computing platform comprising:
at least one textile including a plurality of nodal regions for interfacing with one or more anatomical features of a user, the respective nodal regions configured to be in communication with at least one nearby nodal region;
at least one computing node positioned substantially within one of the textile nodal regions, the at least one computing node including at least one sensory device; and
a gateway device coupled to the plurality of textile nodal regions, the gateway device including:
a gateway processor; and
a memory coupled to the gateway processor and storing processor-executable instructions that, when executed by the gateway processor, configure the gateway processor to:
transmit, via a textile communication network, a discovery signal and a network time protocol signal;
receive node discovery data from one or more computing nodes to enumerate a subset of computing nodes for a current textile system state;
generate physiological analytics based on a series of sensory data records received from the enumerated subset of computing nodes for the current textile system state, the respective sensory data records associated with a time-stamp based on the network time protocol signal; and
transmit actuating signals to the one or more enumerated subset of computing nodes based on the physiological analytics for generating feedback to a user on a substantially real time basis.

2. The textile computing platform of claim 1, wherein the enumerated subset of computing nodes include one or more actuating devices, and wherein the transmitted actuating signals are configured to generate a combination output at the one or more actuating devices.

3. The textile computing platform of claim 1, wherein the node discovery data includes data records identifying a sensor device type and a sensor position relative to an anatomical feature of the user.

4. The textile computing platform of claim 3, wherein the processor-executable instructions, when executed by the gateway processor, configure the gateway processor to:
determine a subset of physiological analytics that correspond to at least one of the identified sensor device type or a sensor position,
wherein the subset of physiological analytics is based on capabilities of sensory data records associated with the identified sensor device type or sensor position.

5. The textile computing platform of claim 1, wherein the physiological analytics includes at least one of gait analysis, biofeedback training, auto-coaching operations in substantial real-time, gamification operations, health monitoring, or medical diagnosis operations.

6. The textile computing platform of claim 1, wherein the series of sensory data records include at least one of inertial measurement unit data, temperature or heat flux data at a skin surface of the user, or electrical biosignals across a skin surface of the user.

7. The textile computing platform of claim 1, wherein the plurality of computing nodes are positioned at positions across the anatomical features of the user for a defined physiological analytics type.

8. The textile computing platform of claim 1, wherein the plurality of computing nodes are positioned across at least one of a foot of the user, a position proximal to a knee of the user, or a position proximal upper leg or lower leg muscles of the user for generating gait analysis for the user.

9. The textile computing platform of claim 1, wherein the at least one computing nodes respectively include:
a node processor;
the at least one sensory-actuating device coupled to the node processor; and
a node memory coupled to the processor and storing processor-executable instructions that, when executed by the node processor, configure the node processor to:
generate sensory data records based on the at least one sensory-actuating device and associating a time-stamp based on a received network time protocol signal;
receive one or more actuating signals for generating sensory-actuating output for application across an anatomical feature of the user as a combination output of the enumerated subset of computing nodes.

10. The textile computing platform of claim 9, wherein the at least one sensory-actuating device is configured to provide at least one of haptic output, heating or cooling output, electrical stimulation, or acoustic output across the anatomical feature of the user.

11. The textile computing platform of claim 9, wherein the node memory includes processor-executable instructions that, when executed by the node processor, configure the node processor to:
generate physiological analytic portions based on the sensory data records generated at the present computing node, the physiological analytic portions being subset analytics for generating physiological analytics based on a combination of computing nodes of the textile computing system.

12. The textile computing platform of claim 1, wherein the textile communication network includes a combination of a conductive fibers integral to the at least one textile and wireless communication circuits for wireless communication.

13. The textile computing platform of claim 1, wherein at least one of the plurality of nodal regions includes a nodal receptacle adapted to removably receive a discrete computing node therein.

14. The textile computing platform of claim 1, wherein at least one computing node includes a power source coupled to the node processor, and wherein the textile communication network is configured to transmit power to adjacent computing nodes associated with nearby nodal regions.

15. A method for a textile computing system including at least one textile for interfacing with one or more anatomical features of a user and at least one computing node including a sensory device positioned on the at least one textile, the method comprising:
transmitting, via a textile communication network, a discovery signal and a network time protocol signal;
receiving node discovery data from one or more computing nodes to enumerate a subset of computing nodes for a current textile system state;
generating physiological analytics based on a series of sensory data records received from the enumerated subset of computing nodes for the current textile system state, the respective sensory data records associated with a time-stamp based on the network time protocol signal; and
transmitting actuating signals to the one or more enumerated subset of computing nodes based on the physiological analytics for generating feedback to a user on a substantially real time basis.

16. The method of claim 15, wherein the enumerated subset of computing nodes include one or more actuating devices, and wherein the transmitted actuating signals are configured to generate a combination output at the one or more actuating devices.

17. The method of claim 15, wherein the node discovery data includes data records identifying a sensor device type and a sensor position relative to an anatomical feature of the user.

18. The method of claim 17, comprising:
determine a subset of physiological analytics that correspond to at least one of the identified sensor device type or a sensor position,
wherein the subset of physiological analytics is based on capabilities of sensory data records associated with the identified sensor device type or sensor position.

19. The method of claim 15, the plurality of computing nodes are positioned across at least one of a foot of the user, a position proximal to a knee of the user, or a position proximal upper leg or lower leg muscles of the user for generating gait analysis for the user.

20. The method of claim 15, wherein the series of sensory data records include at least one of inertial measurement unit data, temperature or heat flux data at a skin surface of the user, or electrical characteristics across a skin surface of the user.
